(19) United States
(12) Patent Application Publication
(10) Pub. No.: US 2026/0199702 A1
(43) Pub. Date: Jul. 16, 2026

(54) MULTIFUNCTIONAL HEALTHCARE MASSAGE, DEODORIZING AND STERILIZING COMB

(71) Applicant: Feng Ze ZHANG, Hsinchu City (TW)

(72) Inventor: Feng Ze ZHANG, Hsinchu City (TW)

(21) Appl. No.: 19/407,417

(22) Filed: Dec. 3, 2025

(30) Foreign Application Priority Data
Jan. 16, 2025 (TW) 114101919

(51) Int. Cl. A61N 5/06; A01K 13/00; A45D 24/10; A61N 1/32

(52) U.S. Cl. CPC A61N 5/0624; A01K 13/002; A45D 24/10; A61N 1/322; A45D 2200/205; A61N 2005/0644; A61N 2005/0651; A61N 2005/066; A61N 2005/0661

(57) ABSTRACT

A multifunctional healthcare massage, deodorizing and sterilizing comb includes a comb body and a comb handle. In pet-type comb, ozone generator is disposed at bottom of comb body, with comb handle having therein a control board, battery, and charging port, whereas UVA LED, Far-Infrared (FIR) LED, and comb teeth are mounted on surface of comb body. In human-type comb, anion generator and electrical muscle stimulation (EMS)-oriented microcurrent generator are disposed at bottom of the comb body, with comb handle having therein a control board, battery, and charging port, whereas anion outlet, FIR LED, and comb teeth are mounted on surface of comb body. When used on pets, the invention performs deodorization, sterilization, and FIR irradiation to promote blood circulation and enhance immunity. When used on humans, the invention promotes blood circulation through FIR irradiation, performs massage through pulsed microcurrent, and achieves deodorization through attachment of anions to hair.

### BACKGROUND OF THE INVENTION

### 1. Field of the Invention

The invention relates to a multifunctional healthcare massage, deodorizing and sterilizing comb and more particularly to a comb structure comprising a comb body and a comb handle. In the pet-type comb, a comb teeth base of comb teeth of the comb body has therein an ozone (O3) generator, and the comb handle has therein a control board, a battery, and a charging port, wherein the surface of the comb body is provided on both sides of its central portion with an Ultraviolet A (UVA) LED, a Far-Infrared (FIR) LED, and the comb teeth. In the human-type comb, a comb teeth base of the comb teeth of the comb body has therein an anion generator and an electrical muscle stimulation (EMS)-oriented microcurrent generator, and the comb handle has therein a control board, a battery, and a charging port, with the comb tooth casing surface being provided with an anion outlet, a FIR LED, and the comb teeth.

### 2. Description of Related Art

With the rapid advancement of technology, modern combs have transcended their traditional role as mere grooming tools, evolving into sophisticated multifunctional devices. However, most existing multifunctional combs typically offer only a single auxiliary feature—such as scalp massage, light therapy, or antibacterial treatment. As a result, comb products have gradually expanded into the realms of medical care, serving the needs of both humans and pets. The development direction of current multifunctional combs therefore focuses on integrating multiple therapeutic and healthcare functions into a single comb structure.

### SUMMARY OF THE INVENTION

It is therefore one object of the invention to provide a multifunctional healthcare massage, deodorizing and sterilizing comb, comprising a comb body and a comb handle. In the pet-type comb, an ozone (O3) generator is disposed at the bottom of the comb body, with the comb handle having therein a control board, a battery, and a charging port, whereas an Ultraviolet A (UVA) LED, a Far-Infrared (FIR) LED, and comb teeth are mounted on the surface of the comb body. In the human-type comb, an anion generator and an electrical muscle stimulation (EMS)-oriented pulsed microcurrent (PMC) generator are disposed at the bottom of the comb body, with the comb handle having therein a control board, a battery, and a charging port, whereas an anion outlet, Far-Infrared LEDs, and comb teeth are mounted on the surface of the comb body.

Applications of the multifunctional healthcare massage, deodorizing and sterilizing comb of the invention are as follows:
1. When the multifunctional pet-type comb is used for grooming pets, ozone is emitted and adheres to the pet's hair for deodorization and sterilization. UVA assists in sterilization and effectively reduces skin abnormalities. Far-infrared (FIR) irradiation promotes blood circulation and improves the health of the pets.
2. When the multifunctional human-type comb is used, scalp massage-oriented pulsed microcurrent and Far-Infrared (FIR) irradiation together promote scalp blood circulation and relieve local tension. Negative ions (i.e., anions) released during combing attach to hair to effectively remove odor.

The invention is effective in deodorizing, sterilizing, promoting blood circulation, and enhancing immunity of pets through Far-Infrared (FIR) irradiation. The invention is further effective in promoting blood circulation of human beings through Far-Infrared (FIR) irradiation, massaging the human scalp with pulsed microcurrent, and attaching anions to the human hair to achieve deodorization. In short, the main functions of the invention are as follows: ozone deodorization and sterilization, UVA sterilization, Far-Infrared (FIR) irradiation, anion generation, and EMS-oriented PMC generation.

The above and other objects, features and advantages of the invention will become apparent from the following detailed description taken with the accompanying drawings.

### BRIEF DESCRIPTION OF THE DRAWINGS

FIG. 1 is a lateral cross-sectional view of a multifunctional healthcare massage, deodorizing and sterilizing comb of the invention;

FIG. 2 is a schematic view of a comb body of a multifunctional healthcare massage, deodorizing and sterilizing comb of the invention;

FIG. 3 is a schematic view of buttons disposed on a comb handle of a multifunctional healthcare massage, deodorizing and sterilizing comb of the invention;

FIG. 4 is another lateral cross-sectional view of a multifunctional healthcare massage, deodorizing and sterilizing comb of the invention;

FIG. 5 is another schematic view of the comb body of a multifunctional healthcare massage, deodorizing and sterilizing comb of the invention; and

FIG. 6 is another schematic view of buttons disposed on a comb handle of a multifunctional healthcare massage, deodorizing and sterilizing comb of the invention.

### DETAILED DESCRIPTION OF THE INVENTION

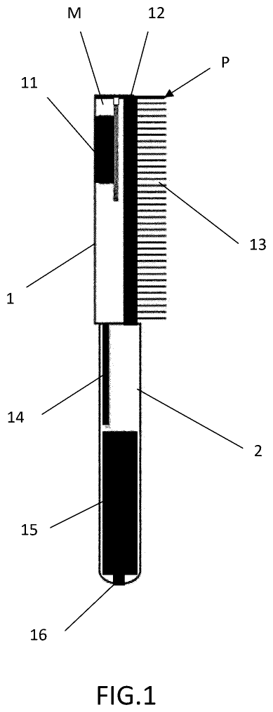

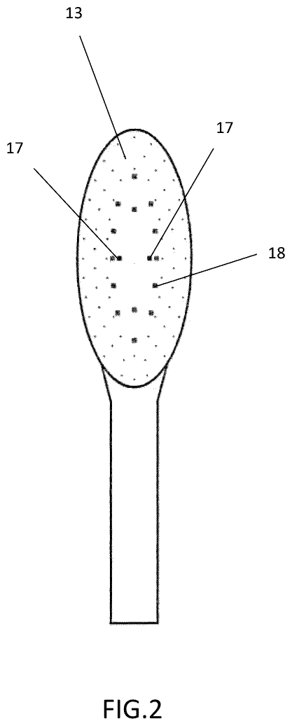

The invention is a multifunctional healthcare massage, deodorizing and sterilizing comb. Referring to FIGS. 1 to 2, a pet-type comb P comprises a comb body 1 and a comb handle 2. A comb teeth base M of comb teeth 13 of the comb body 1 has therein an LED circuit board 12 and an ozone generator 11. The comb handle 2 has therein a control board 14, a battery 15, and a charging port 16. The surface of the comb body 1 is provided on both sides of its central portion with Ultraviolet A (UVA) LEDs 17, Far-Infrared (FIR) LEDs 18, and the comb teeth 13. The FIR LEDs 18 are elliptically arranged on a central surface of the comb body 1. The UVA LEDs 17 are disposed on both sides of the central surface of the comb body 1.

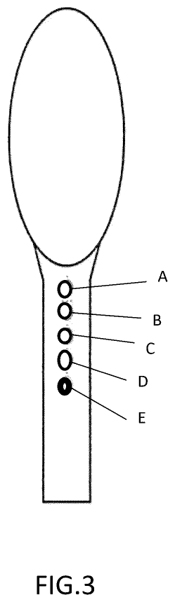

Referring to FIG. 3, a main power button A, an ozone (O3) button B, a UVA button C, a UVA+ozone combined button D, and an FIR button E are arranged sequentially from bottom to top on the comb handle of a multifunctional healthcare massage, deodorizing and sterilizing comb.

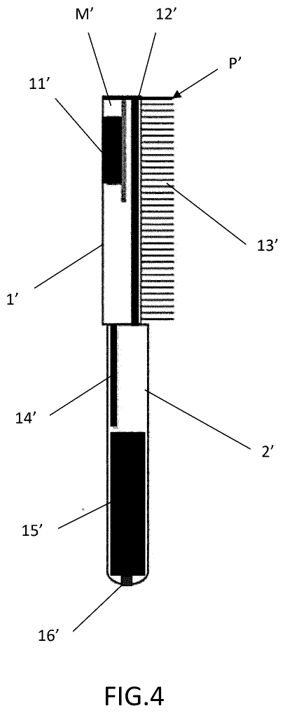

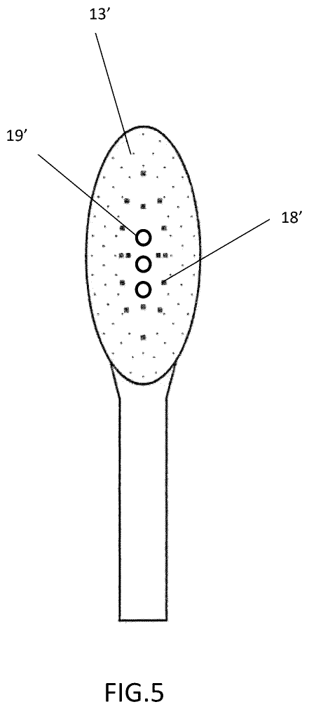

Referring to FIGS. 4 to 5, a human-type comb P′ comprises a comb body 1′ and a comb handle 2′. A comb teeth base M′ of comb teeth 13′ of the comb body 1′ has therein an LED circuit board 12′, an anion generator 11′, and an EMS-oriented PMC generator 17′. The comb handle 2′ has therein a control board 14 , a battery 15′, and a charging port 16′. An anion outlet 19′, Far-Infrared (FIR) LEDs 18′, and comb teeth 13′ are centrally mounted on the surface of the comb body 1′. The Far-Infrared LEDs 18′ are elliptically arranged on the central surface of the comb body 1′.

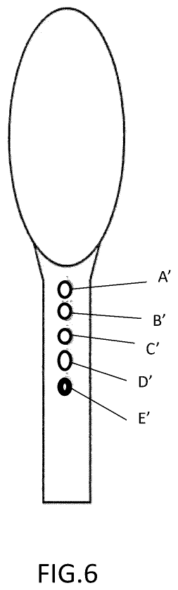

Referring to FIG. 6, a main power button A′, an anion button B′, a Far-Infrared (FIR) button C′, an EMS-oriented PMC button D′, and a PMC+Far-Infrared combined button E′ are arranged sequentially from bottom to top on the comb handle of a multifunctional healthcare massage, deodorizing and sterilizing comb.

While the invention has been described in terms of preferred embodiments, those skilled in the art will recognize that the invention can be practiced with modifications within the spirit and scope of the appended claims.

What is claimed is:

1. A multifunctional healthcare massage, deodorizing and sterilizing comb, being a pet-type comb, comprising:
a comb body; and
a comb handle,
wherein a comb teeth base of comb teeth of the comb body has therein an LED circuit board and an ozone generator,
wherein the comb handle has therein a control board, a battery, and a charging port; wherein a surface of the comb body is provided on both sides of its central portion with Ultraviolet A (UVA) LEDs, Far-Infrared (FIR) LEDs, and the comb teeth.

2. The multifunctional healthcare massage, deodorizing and sterilizing comb of claim 1, wherein the Far-Infrared LEDs are elliptically arranged on a central surface of the comb body, and the Ultraviolet A LEDs are disposed on both sides of the central surface of the comb body.

3. The multifunctional healthcare massage, deodorizing and sterilizing comb of claim 1, wherein a main power button, an ozone (O3) button, a UVA button, a UVA+ozone (O3) combined button, and a FIR button are arranged sequentially from bottom to top on the comb handle.

4. A multifunctional healthcare massage, deodorizing and sterilizing comb, being a human-type comb, comprising:
a comb body; and
a comb handle,
wherein a comb teeth base of comb teeth of the comb body has therein an LED circuit board, an anion generator, and an electrical muscle stimulation (EMS)-oriented microcurrent generator,
wherein the comb handle has therein a control board, a battery, and a charging port; wherein an anion outlet, Far-Infrared LEDs, and the comb teeth are centrally mounted on the surface of the comb body.

5. The multifunctional healthcare massage, deodorizing and sterilizing comb of claim 4, wherein the Far-Infrared LEDs are elliptically arranged on a central surface of the comb body.

6. The multifunctional healthcare massage, deodorizing and sterilizing comb of claim 4, wherein a main power button, an anion button, a FIR button, an EMS-oriented PMC button, and a PMC+FIR combined button are arranged sequentially from bottom to top on the comb handle.
